(19) United States
(12) Patent Application Publication
(10) Pub. No.: US 2010/0331828 A1
(43) Pub. Date: Dec. 30, 2010

(54) HAND-HELD MEDICAL INSTRUMENT

(76) Inventors: Thorsten Pöschel, Potsdam (DE); Thomas Schwager, Berlin (DE); Georg Duda, Berlin (DE); Clara Saluena, Sitges (ES)

Correspondence Address: SMITH PATENT OFFICE, 1901 PENNSYLVANIA AVENUE N W, SUITE 901, WASHINGTON, DC, 20006, US

(21) Appl. No.: 12/741,069

(22) PCT Filed: Oct. 31, 2008
(86) PCT No.: PCT/DE2008/001789

§ 371 Date: Aug. 27, 2010

(30) Foreign Application Priority Data
Nov. 1, 2007 (DE) 10 2007 052 539.9

(51) Int. Cl. A61B 17/00

(52) U.S. Cl. 606/1

(57) ABSTRACT

The invention relates to a hand-held medical instrument, in particular a hand-held surgical or dental instrument, comprising a component that generates mechanical vibrations during operation, and a particle damping element which has at least one hollow space that is designed to receive the mechanical vibrations and is filled at least in part with a plurality of granular particles which dampen the mechanical vibrations by means of dissipative shocks between the granular particles and/or between the granular particles and a wall surrounding the hollow space.

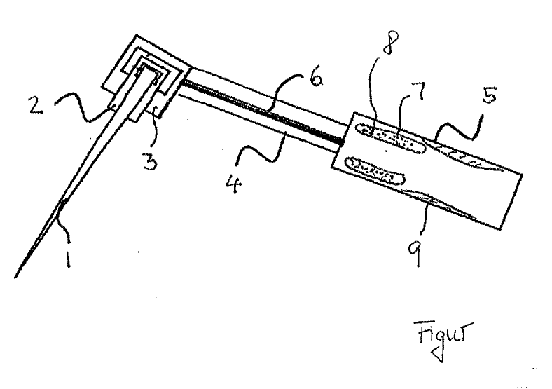

The invention relates to a hand-held medical instrument, in particular a hand-held surgical or dental instrument, comprising a component that generates mechanical vibrations during operation.

### BACKGROUND OF THE INVENTION

Such hand-held medical instruments are instruments which—in use—the user, i.e. a medical practitioner or other medical staff, holds and guides with his hand. During operation, a component of these hand-held medical instruments is moved by means of a mechanical actuator such that vibrations are generated. This is for example the case with surgical bone burs or bone saws as well as hand-held dental instruments such as dental drills and smoothers. During operation, the movements of the component are typically characterized by a high frequency. The mechanical vibrations or oscillations generated in the course of this process are transmitted to the hand of the user. They are not only perceived as unpleasant but can even lead to job-related medical conditions of the user when the instrument is often used over longer periods of time. Furthermore, the vibrations caused by the medical instrument may be perceived as unpleasant or painful by the patient, for example the dentist's patient.

### SUMMARY OF THE INVENTION

The object of the invention is to provide a hand-held medical instrument, in particular a hand-held surgical or dental instrument in which the drawbacks occurring due to the use of components generating mechanical vibrations are avoided or at least minimized.

According to the invention, a hand-held medical instrument, in particular a hand-held surgical or dental instrument according to the independent claim 1 is provided. Advantageous implementations of the invention are the subject matter of the dependent claims.

The invention encompasses the idea of a hand-held medical instrument, in particular a hand-held surgical or dental instrument, comprising a component that generates mechanical vibrations during operation, and a particle damping element which has at least one hollow space that is designed to receive the mechanical vibrations and is filled at least in part with a plurality of granular particles which dampen the mechanical vibrations by means of dissipative shocks between the granular particles and/or between the granular particles and a wall surrounding the hollow space. The mechanical vibrations generated by the component during operation are damped by means of the particle damping element by the plurality of granular particles colliding dissipatively with each other and with the wall. In the course of this process, the energy of the mechanical oscillation is converted either into heat or into harmless low-frequency mechanical oscillations. The handling properties of the medical instrument are in general improved for the user. Occupational illnesses are avoided. Additionally, the damping of the mechanical oscillations makes it easier for the user to guide the hand-held medical instrument properly and calmly during application such that the intended purpose can be served better and possible pain felt by the patient is minimized. The particles can also be referred to as damping particles.

A preferred further development of the invention provides for the component being a treatment component. In the case of a hand-held surgical instrument, the treatment component is a saw blade or a bone bur, for example. In the case of a hand-held dental instrument, the treatment component can be implemented as a dental drill or smoother. These treatment components are in all the examples machining treatment components. The actuator in the hand-held medical instrument can be implemented by means of an electrical or pneumatic actuation means which is not limited to these types of components. In this connection, different actuation types are known which are optimized for the respective application, depending on the hand-held medical instrument. The hand-held medical instruments in the different embodiments have in common that mechanical vibrations are generated due to the actuated movement of one or more components which vibrations are then damped with the particle damping element.

In a convenient variant of the invention, it can be provided for the component being a component generating high-frequency mechanical vibrations during operation. The particle damping element is then designed to damp high-frequency oscillations.

An advantageous embodiment of the invention provides for the particle damping element being designed to keep a handle portion substantially free from vibrations. Through this, the transmission of the mechanical oscillations to the hand of the user is particularly efficiently minimized or completely eliminated. Preferably, a further development of the invention provides for the at least one hollow space being formed within the region of the handle portion.

A further development of the invention can provide for the granular particles being made of a material selected from the following group of materials: sand, glass, plastic and metal, for example lead. Conveniently, spheres are provided as granular particles. The plurality of the granular particles in one hollow space can be made of one material. Alternatively, it can be provided for the plurality of particles in one hollow space containing particles made of two or more materials. Additionally, an implementation can provide for several hollow spaces being formed on the hand-held medical instrument which are filled with charges of granular particles of varying types. In this connection, it can for example be provided for one hollow space being filled with granular particles made of one material and another hollow space being filled with particles made of another material. In this way, the configuration options for the damping properties are extended to thus react on different intended purposes.

A preferred further development of the invention provides for the granular particles having a diameter of about 0.05 mm to about 2 mm. The granular particles in one hollow space may have a uniform size or different sizes within a customary, typically manufacturing-related distribution range. Additionally, it can be provided for filling only granular particles having a first size into one hollow space and filling only granular particles having another size into another hollow space, the other size differing from the first size.

In a further development of the invention, it can be provided for built-in elements being arranged in the hollow space. These are structures, for example guiding plates or protrusions which extend into the hollow space and thus influence the collision behaviour of the granular particles.

### DESCRIPTION OF A PREFERRED EXEMPLARY EMBODIMENT OF THE INVENTION

In the following, the invention is explained in more detail using an exemplary embodiment with reference to a FIGURE.

The only FIGURE shows a schematic illustration of a hand-held medical instrument in the form of a device for the treatment of teeth. A dental component 1 for the treatment of teeth or tooth surfaces is clamped into a receptacle 2. The dental component 1 is implemented as a dental file, a smoother or a dental drill, for example, which in particular consists of a high-strength material such as titanium. The receptacle 2 substantially corresponds to a drill chuck into which the dental component 1 for the transmission of torques is removably clamped.

The receptacle 2 is pivoting in a holder 3. The holder 3 is rigidly coupled with a drive apparatus 5 via an extension 4. The drive apparatus 5 is connected with the pivoting receptacle 2 via a pivoting shaft 6 to actuate the receptacle 2. Within the region of the holder 3, a cogwheel construction, for example, is provided for this purpose. The drive apparatus 5 is preferably formed by an electric motor which is coupled with the shaft 6 in a torque-proof manner, however, it may also be replaced by a pneumatic drive apparatus, for example. Furthermore, a direct actuation of the receiving appliance 2 by means of the drive apparatus 5 is provided alternatively.

During operation of the dental component 1, mechanical vibrations are generated which normally have a frequency that corresponds with the motion frequency of the dental component 1. Hollow spaces 7 are formed on the extension 4 which are in part filled with granular particles 8. Thus, a particle damping element is formed which damps the mechanical vibrations. Alternatively or additionally, hollow spaces with a charge (not shown) may be formed within the region of a handle portion 9. The hollow spaces having a granular particle charge and surrounded by the particle damping element may be arranged on the outside of the housing of the hand-held medical instrument and/or may be integrated into this housing.

The features of the invention disclosed in the above description, the claims and the FIGURE can be of importance both taken on their own and in any combination to implement the invention in its different embodiments.

1. A hand-held medical instrument, in particular a hand-held surgical or dental instrument, comprising a component that generates mechanical vibrations during operation, and a particle damping element which has at least one hollow space that is formed to receive the mechanical vibrations and is filled at least in part with a plurality of granular particles which dampen the mechanical vibrations of dissipative shocks between the granular particles and/or between the granular particles and a wall surrounding the hollow space.

2. The hand-held instrument according to claim 1, wherein the component is a treatment part.

3. The hand-held instrument according to claim 1, wherein the component is a component generating high-frequency mechanical vibrations during operation.

4. The hand-held instrument according to claim 1, wherein the particle damping element is designed to keep a handle portion substantially free from vibrations.

5. The hand-held instrument according to claim 4, wherein the at least one hollow space is formed within the region of the handle portion.

6. The hand-held instrument according to claim 1, wherein the granular particles are made of a material selected from the following group of materials: sand, glass, plastic and metal.

7. The hand-held instrument according to claim 1, wherein the granular particles have a diameter of about 0.05 mm to about 2 mm.

8. The hand-held instrument according to claim 1, further comprising built-in elements arranged in the hollow space.
